(19) United States
(12) Patent Application Publication
(10) Pub. No.: US 2010/0160625 A1
(43) Pub. Date: Jun. 24, 2010

(54) Novel Preparation of 6-O-Acyl Chlorosucrose from Anhydrous Cholorinated Reaction Mass

(76) Inventors: Rakesh Ratnam, Karnataka (IN); Sundeep Aurora, Karnataka (IN); Srikanth Kulkarni, Karnataka (IN)

Correspondence Address: THE NATH LAW GROUP, 112 South West Street, Alexandria, VA, 22314, US

(21) Appl. No.: 11/992,431

(22) PCT Filed: Sep. 21, 2006
(86) PCT No.: PCT/IN2006/000386

§ 371 Date: Jul. 3, 2008

(30) Foreign Application Priority Data
Sep. 22, 2005 (IN) 1176/MUM/2005

(51) Int. Cl. C07H 1/08

(52) U.S. Cl. 536/128

(57) ABSTRACT

A process is described for production of a chlorinated sucrose from a process stream containing a 6-O-protected chlorinated sucrose derived from chlorination of 6-O-protected sucrose wherein the process stream is treated under conditions which prevent or reverse deacylation of 6-O-protected chlorinated sucrose, extracting the same in a solvent, washing most of the dimethylformamide free from the solvent extract by repeated washing with saturated sodium chloride solution, isolating the 6-O-protected sucrose as a pure fraction and obtaining a chlorinated sucrose by deacylating the same.

### TECHNICAL FIELD

The present invention relates to a novel process and a novel strategy for production of 1′-6′-Dichloro-1′-6′-DIDEOXY-β-Fructofuranasyl-4-chloro-4-deoxygalactopyranoside (TGS) involving preparation of a chlorinated reaction mass exclusively in 6-O-protected form and no residual TGS. TGS in its 6-O-protected form can be extracted and isolated in a easier way compared to TGS.

### BACKGROUND OF THE INVENTION

Strategies of prior art methods of production of TGS predominantly involve chlorination of 6-O-protected sucrose by use of Vilsmeier Haack reagent derived from various chlorinating agents such as phosphorus oxychloride, oxalyl chloride, phosphorus pentachloride etc, and a tertiary amide such as dimethyl formamide (DMF) to chlorinate 6-O-protected Sucrose, to form 6 acetyl 4,1′,6′trichlorogalactosucrose. After the said chlorination reaction, the reaction mass is neutralized to pH 7.0-7.5 using appropriate alkali hydroxides of calcium, sodium, etc. and pH increased further to 9.5 or above to deacylate/deacetylate the 6-acetyl 4,1′,6′trichlorogalactosucrose to form 4,1′,6′trichlorogalactosucrose, the TGS.

In above scheme, purification of TGS from the final reaction mixture is a more difficult job than an alternative process wherein 6-O-protected TGS is extracted in an organic solvent from the reaction mass, isolated, purified and then subjected to deacylation. However, in this option, in prior art process, during the chlorination, the reaction mixture is undergoing, slow deacylation of 6-O-protected TGS continues to progress to form a significant quantity of TGS. This TGS formed during the reaction after neutralization and filtration requires a very high quantity of a solvent for its extraction. Thus the neutralized mass after chlorination should contain almost no TGS to facilitate better extraction and no loss of TGS.

### SUMMARY OF THE INVENTION

A process is described for production of a chlorinated sucrose compound from a process stream containing a 6-O-protected chlorinated sucrose derived from chlorination of 6-O-protected sucrose wherein the process stream is neutralized by a mild alkali, preferably by gaseous ammonia, maintaining pH to about 5-6, acylating the TGS formed during the process of neutralization by adding acetic anhydride and holding for a period enough for disappearance of most of the TGS thus formed, extracting the 6-O-protected sucrose in a solvent, washing DMF free from the process stream by repeated washing with saturated sodium chloride solution, isolating the 6-O-protected sucrose as a pure fraction and obtaining a chlorinated sucrose by deacylating the same.

### DETAILED DESCRIPTION OF THE INVENTION

TGS, in its 6-O-protected form, is easier to extract in water immiscible solvents such as ethyl acetate, chloroform, methyl ethyl ketone, etc. This makes it strategically more reasonable if its deblocking is not done at the neutralization step. It is also more useful to make the removal of DMF further down during the processing which means that it is advantageous to maintain TGS presence in 6-O-acyl form. In the invented process, the deblocking of 6-O-acyl TGS is carried out after it is totally isolated from the reaction mixture/process stream by a process step including solvent extraction.

However, during the chlorination reaction, due to the elevated temperatures and highly acidic nature of the reaction mass, the deacylation at the 6^{th} position does proceed slowly, nevertheless. This deacylated product appears as TGS directly after neutralization. Amount of such prematurely deacylated product, however, is below 10% of the total reaction mass.

The chlorinated reaction mass is then neutralized with a suitable base. The neutralization has to be in a well controlled manner if the compound TGS should be in its 6-O-protected form. If the pH of the neutralized mass crosses 7.0, the deacylation takes place slowly and the TGS gets formed.

Use of the mild alkali during the neutralization and maintaining the pH below 5.5 results helps in achieving and in maintaining majority of the product as 6-O-acyl TGS. However, 10% to 25% of the TGS, despite these precautions, slowly gets inevitably deacylated during the neutralization step. This much amount of the deacylated compound present in the neutralized reaction mass does not get completely extracted from the mass and results in enormous consumption of the extraction solvent such as ethyl acetate, butyl acetate, etc.

This invention describes an innovative process wherein neutralization of the reaction mass is done under anhydrous conditions and the reaction mass is further mildly acylated with an acylating agent to protect again the reactive 6^{th} position. This enables the acylation of any residual TGS formed during the neutralization of the chlorinated reaction mass.

After the chlorination reaction using the Vilsmeier reaction using the tertiary amide, the chlorination is terminated by sparging ammonia gas in the reaction flask. This is accompanied by addition of 0.1 to 0.5 volumes of the tertiary amide such as dimethyl formamide into the reaction mass optionally buffered with ammonium acetate. The pH of the reaction mass was adjusted to 5-6.

The reaction mass is then cooled with stirring up to 0° C. An acid anhydride such as acetic anhydride, diluted 1:2 to 1:4 times using the tertiary amide such as Dimethylformamide is added dropwise to the reaction mass and temperature is controlled below 8° C.

The absence of TGS formed during the anhydrous neutralization using ammonia can be checked by TLC. During neutralization due to some spot heat generation some of the 6-O-Acyl derivative is converted to TGS, which is converted back to the 6-O-acyl derivative by adding limited quantity of an acylating agent like acetic anhydride and this acylating reaction is terminated by adding 1:1 volume of demineralized water.

The reaction mass containing the 6-O-acyl TGS can be taken up for further purification by solvent extraction and isolation.

Solvents that can be used for extraction of 6-O-protected chlorinated sucrose include one or more of an organic solvent comprising ethyl acetate, butyl acetate, methyl ethyl ketone, methylene chloride, ethylene dichloride, toluene and the like. A significant quantity of DMF gets extracted in the solvent extract in this way, which needs to be washed away by a suitable method. Preferred method used here for this purpose includes repeated washing of the solvent extract with saturated salt solution, the salt preferably being a sodium chloride, until content of DMF gets reduced considerably, preferably to 0.5% or less. Of course, any other method of DMF removal can potentially be used within the scope of this invention.

The purified extract in the solvent can then be subjected to isolation of the 6-O-protected chlorinated sucrose to be used further for deacylation by a method of choice for production of a chlorinated sucrose. The chlorinated sucrose of the preferred invention is trichlorogalactosucrose and the preferred 6-O-protected chlorinated sucrose is 6-O-acetyl TGS or 6-O-benzoyl TGS. The invention, however, covers within its scope one or more of other chlorinated sucrose too and one or more of an other protecting acyl group too.

Embodiments of a process stream/a chlorination reaction mixture which can be subjected to the process described in this invention include, a process stream generated after chlorination step described in Mufti et al. (1983) U.S. Pat. No. 4,380,476, Walkup et al. (1990 U.S. Pat. No. 4,980,463), Jenner et al. (1982) U.S. Pat. No. 4,362,869, Tulley et al. (1989) U.S. Pat. No. 4,801,700, Rathbone et al. (1989) U.S. Pat. No. 4,826,962, Bornemann et al. (1992) U.S. Pat. No. 5,141,860, Navia et al. (1996) U.S. Pat. No. 5,498,709, Simpson (1989) U.S. Pat. No. 4,889,928, Navia (1990) U.S. Pat. No. 4,950,746, Neiditch et al. (1991) U.S. Pat. No. 5,023,329, Walkup et al. (1992) U.S. Pat. No. 5,089,608, Dordick et al. (1992) U.S. Pat. No. 5,128,248, Khan et al. (1995) U.S. Pat. No. 5,440,026, Palmer et al. (1995) U.S. Pat. No. 5,445,951, Sankey et al. (1995) U.S. Pat. No. 5,449,772, Sankey et al. (1995) U.S. Pat. No. 5,470,969, Navia et al. (1996) U.S. Pat. No. 5,498,709, Navia et al.(1996) U.S. Pat. No. 5,530,106

Described in the following are examples, which illustrate working of this invention without limiting the scope of this invention in any manner. Reactants, proportion of reactants used, range of reaction conditions described, and the like are only illustrative and the scope extends to their analogous reactants, reaction conditions and reactions of analogous generic nature. In general, any equivalent alternative which is obvious to a person skilled in art of chlorinated sucrose production is covered within the scope of this specification. Thus, mention of “an acetate” covers any equivalent acyl group which can perform the same function in the contest of this invention. A mention of “a chlorinated sucrose compound” includes, in addition to preferred embodiments of trichlorogalactosucose and sucrose-6-acetate or benzoate, any of the chlorinated sucrose or an acyl derivative of sucrose to which the process of invention is applicable, and all of them are included in the claim. Several other adaptations of the embodiments will be easily anticipated by those skilled in this art and they are also included within the scope of this specification. Mention in singular is construed to cover its plural also, unless the context does not permit so, viz: use of “an organic solvent” for extraction covers use of one or more of an organic solvent in succession or in a combination as a mixture.

### EXAMPLE 1

### Preparation of TGS by Prior Art Process

In a 5 L reaction flask, placed 1280 ml of Dimethylformamide and cooled to 0-5° C. Then added 635 g of Phosphorous pentachloride (5.4 moles) slowly under stirring, maintaining the temperature of the reaction mass below 30° C. The Vilsmeier was allowed to form and the mass is further cooled to below 0° C. and the sucose-6-acetate (200 g equivalent) in DMF solution is added slowly at 0-5° C. Then the reaction mass is allowed to attain 25-30° C. and stirred for 60 minutes and is heated to 80° C. and held for 1 hour, further heated to 100° C. and held for 6 hours and finally at 110-115° C. and held for 2-3 hours. The progress of the reaction is monitored by HPLC analysis. The TGS content obtained was 42% of sucrose-6-acetate input.

### EXAMPLE 2

### Quenching of Chlorinated Reaction Mass Using Ammonia Gas

In an experiment, 2.2 L of chlorinated reaction mass containing 75 g of TGS equivalent was taken for quenching using ammonia gas under anhydrous conditions. The reaction mass was cooled to 0-5° C. temperature. Ammonia gas was connected to the sparger line of the reaction flask and slowly bubbled through the reaction mass. 150 ml of DMF was added slowly to the reaction contents. The mass was kept under stirring and pH was adjusted up to 5.8. Approximately 185 g of ammonia gas was consumed for carrying out the process. The amount of deacylated TGS was found to be 15% by HPLC in the reaction mass after the above said quenching process.

### EXAMPLE 3

Acetylation of Deacetylated Residues of TGS in Quenched Mass

The mass from Example 2 was then held cold at 0° C. and 35.8 g of acetic anhydride diluted with 1:2 times v/v with DMF was added dropwise with continuous stirring. The acylation reaction was continued for a period of 3.0 hrs. The disappearance of deacylated TGS was monitored by TLC. Also the formation of other acetates was also controlled.

The reaction mass after 3.0 hrs showed TGS content of less than 1%. The reaction was terminated by adding 1.8 L of demineralized water below 8° C. The final pH of the reaction mass was found to be 6.0.

### EXAMPLE 4

### Extraction of 6-O-acetyl TGS and Further Isolation of TGS

The 6-O-acetyl TGS from the reaction mass (volume—4 L) obtained from example 3 was extracted with 1:3 times of ethyl acetate and the layers were separated. The ethyl acetate extract was then concentrated to 50% of its initial volume and was washed with 1:0.1 times of saturated sodium chloride solution to remove the DMF from the solution. This washing was repeated up to 15 times for reducing the DMF content to less than 0.5%.

The ethyl acetate layer was then concentrated to thick syrup and then loaded on to silanized silica gel. The pure 6-O-acetyl TGS was eluted out using pH 10.5-11.0 aqueous buffer solution and was concentrated by reverse osmosis membrane.

The concentrated aqueous solution was treated with 20% sodium hydroxide solution till pH 9.0-9.5 was attained and the deacetylation was monitored by TLC. After complete deacetylation, the pH of the solution was adjusted to 7.0 using dilute HCl. The aqueous solution containing 15% TGS was extracted into 1:3.5 times of ethyl acetate.

The ethyl acetate layer was separated, concentrated and TGS crystallized from the solution was filtered and dried. The purity of TGS obtained was 96.3% and the overall yield was found to be 22% of sucrose input.

### EXAMPLE 5

### Isolation of TGS by Prior Art Process

In a 10 L reaction flask, 2.5 L of Dimethylformamide was taken and cooled to 0-5° C. Then added 1270 g of Phosphorous pentachloride (5.4 moles) slowly under stirring, maintaining the temperature of the reaction mass below 30° C. and the Vilsmeier reagent was allowed to form. The mass was then further cooled to below 0° C. and 400 g of sucose-6-acetate solution in DMF was added slowly at 0-5° C. Then the reaction mass was allowed to attain 25-30° C. and stirred for 60 minutes and heated to 80° C., held for 60 minutes, further heated to 100° C. and held for 6 hours and finally at 110-115° C. and held for 2-3 hours. The progress of the reaction is monitored by HPLC analysis. The TGS content obtained was 43.6% of sucrose-6-acetate input.

The chlorinated reaction mass was then neutralized using calcium hydroxide slurry in water up to pH 7.0-7.5. Then the pH was further raised to 9.5 and was kept stirring for 5 hours to complete the deacetylation of 6-acetyl TGS to TGS. The deacetylation was confirmed by TLC analysis. The pH of the mass was then adjusted to neutral by addition of dilute HCl solution. The total volume of the neutralized mass was found to be 18.5 L

The mass was then extracted into 1:3.5 times of ethyl acetate and the layers were separated. The ethyl acetate extract was then concentrated to 50% of its initial volume and was washed with 1:0.1 times of saturated sodium chloride solution to remove the DMF from the solution. This washing was repeated up to 15 times for reducing the DMF content to less than 0.5%.

The ethyl acetate layer was then concentrated to thick syrup and then loaded on to silanized silica gel. The pure TGS was eluted out using pH 10.5-11.0 aqueous buffer solution and was concentrated by reverse osmosis membrane. The aqueous solution containing 15% TGS was extracted into 1:3.5 times of ethyl acetate.

The ethyl acetate layer was separated, concentrated and TGS crystallized from the solution was filtered and dried. The purity of TGS obtained was 97.0% and the

[symbol]

1-4. (canceled)

5. A process of production of a chlorinated sucrose from a process stream comprising purification of 6-O-protected chlorinated sucrose from a solution and its subsequent deacylation and isolation of the chlorinated sucrose wherein the said solution is substantially anhydrous and the said process comprising one or more of following steps:
a. an acidic process stream containing 6-O-protected sucrose having pH below 5 is neutralized under anhydrous condition by a mild alkali preventing the pH to go beyond 7, more preferably between 5 to 6,
b. preferably chlorinated sucrose formed in a process prior to purification of the said anhydrous solution of 6-O-protected chlorinated sucrose, including a process of neutralization of an acidic solution having pH below 5, is acylated by treating with an acylating agent to produce 6-acyl chlorinated sucrose.

6. A process of claim 5 wherein
a. the said chlorinated sucrose compound comprises a chlorinated sucrose including trichlorogalacrosucrose, a dichlorosucrose, a tetrachlorosucrose and the like,
b. the said chlorination reaction comprises reacting sucrose or sucrose derivative with one or more of a chlorinating reagent by one or more of a process including:
i. a reaction of sucrose dissolved in pyridine with sulphuryl chloride, or
ii. a reaction of sucrose with thionyl chloride in a nitrogenous base of free hydroxyl and in presence of non-reactive moderately polar organic solvent, or
iii. a reaction of a 6-O-protected sucrose with a Vilsmeier reagent of a general formula [HCIC=N.sup.+R.sub.2]CI.sup.-, or [HPOCI.sub.2.O.C.sup+=N.sup+.R.sub.2]CI.sup.-, where R represents an alkyl group preferably a methyl or ethyl group,
c. the said mild alkali comprises one or more of an alkali which shall not lead to increase in pH above 7 when added to a solution, including ammonia, preferably in a gaseous form, and the like,
d. the said acylating agent includes acetic anhydride and the like,
e. the said purification of 6-O-protected chlorinated sucrose is preferably done by extraction in a solvent capable of extracting 6-O-protected chlorinated sucrose comprises use of one or more of a partly miscible or immiscible organic solvent including ethyl acetate, butyl acetate, methyl ethyl ketone, methylene dichloride, ethylene dichloride, toluene, and the like,
f. 6-O-protected chlorinated sucrose extracted in one or more of a partly miscible or immiscible organic solvent, is preferably washed with saturated salt solution, preferably a sodium chloride solution to remove dimethylformamide.

7. A process of claim 6 comprising steps of:
a. sparging ammonia gas, as a mild alkali, under anhydrous condition by bubbling through the process stream obtained after a chlorination reaction to achieve its neutralization maintaining pH of the process stream to around 5.8 throughout the neutralization process, preferably with an optional addition of an ammonium acetate buffer,
b. cooling to around 0 degrees celcius and adding acetic anhydride in a quantity and allowing acetylation to occur for a period of time enough to acetylate back most of the TGS formed in the process stream until the end of step (a.) of this claim,
c. terminating the acetylation reaction, preferably by adding demineralized water in 1:1 proportion,
d. extracting 6-O-protected TGS from process stream of step (b.) of this claim by extraction in a solvent, preferably ethyl acetate, and
e. isolating 6-O-protected TGS from the process stream of step (d.) of this claim free from one or more of other extracted constituents by a step of purification and isolation.

8. A process of claim 7 where the said purification in step (d.) comprises removal of dimethylformamide, abbreviated as DMF, by one or more of a step of its removal including repeated washing by saturated salt solution, preferably a sodium chloride solution, until level of DMF in the Process Stream decreases significantly, preferably up to 0.5% or less.
